(19) United States
(12) Patent Application Publication
(10) Pub. No.: US 2025/0073152 A1
(43) Pub. Date: Mar. 6, 2025

(54) MULTIPOTENT HAIR WASHING COMPOSITION

(71) Applicant: Henkel AG & Co. KGaA, Duesseldorf (DE)

(72) Inventors: Camille GROSJACQUES, Hamburg (DE); Volker SCHEUNEMANN, Lueneburg (DE); Katharina Madita BODE, Buxtehude (DE)

(21) Appl. No.: 18/288,581

(22) PCT Filed: Apr. 4, 2022
(86) PCT No.: PCT/EP2022/058870

§ 371 Date: Oct. 27, 2023

(30) Foreign Application Priority Data
May 5, 2021 (DE) 10 2021 111 723.2

(51) Int. Cl. A61K 8/60; A61Q 5/00; A61Q 5/02; A61Q 5/12

(52) U.S. Cl. CPC A61K 8/602; A61Q 5/004; A61Q 5/02; A61Q 5/12; A61K 2800/52; A61K 2800/5922

(57) ABSTRACT

Cosmetic agents may be used for treating a keratin material. The cosmetic agent may include lactobionic acid in an amount ranging from 0.001 to 20 wt. % in relation to the total weight of the cosmetic agent, and the cosmetic agent may further include at least one surfactant in an amount ranging from 0.01 to 40 wt. % in relation to the total weight of the cosmetic agent.

### CROSS-REFERENCE TO RELATED APPLICATIONS

The present application is a national stage entry according to 35 U.S.C. § 371 of PCT application No.: PCT/EP2022/058870 filed on Apr. 4, 2022; which claims priority to German patent application 10 2021 111 723.2 filed on May 5, 2021; all of which are incorporated herein by reference in their entirety and for all purposes.

### TECHNICAL FIELD

The invention relates to cosmetic agents, in particular shampoos and conditioners, having an active ingredient combination for the gentle and effective cleaning or care of hair.

### BACKGROUND

The structure of human hair is affected by external and internal factors. The use of care products, in particular the frequency and intensity of the hair washing, chemical influences and environmental influences (tinting, coloring, hair bleaching, permanent or temporary hair shaping, chlorine water in swimming pools, environmental toxins of different types, etc.), mechanical influences (strong pulling, e.g., by shaping of the hair) and thermal influences (hot blow-drying, permanent wave or else the influence of ultraviolet light, e.g., by exposure to the sun) are to be mentioned in particular as external factors. The internal factors include a balanced and vitamin-rich nutrition, the supply of liquid by sufficient drinking or else possibly the administration of medicaments.

In order to compensate for structural damage to the hair, it has long been customary to subject hair to a treatment using care products. Care products of this type influence the natural structure and properties of hair. Thus, after such treatments, for example, the wet and dry combability of hair, the hold, and the fullness of the hair can be optimized, or the hair can be protected from an increase in split ends.

During treatment, the hair is usually treated with specific active ingredients, for example proteins. Depending on the formulation, the combability, hold, and fullness of the hair can be improved by the treatment, and the damage, the splitting rate and brittleness can be reduced.

Multifunctional cosmetic products which contain care products are known in the prior art. These multifunctional cosmetic products include in particular what are referred to as “2-in-1” shampoos, which not only clean hair but also care for it. Products of this type are highly appreciated by consumers because, due to their product performance, they eradicate the need for at least one process step, for example conditioning with a conventional hair conditioner. These cosmetic agents are often provided primarily as a cleaning agent, which, however, also achieve a care effect and reduce structural damage.

It is also important to restore the hair structure after damage. The common term for restoring or improving the hair structure is the “repair effect.” The success of the repair effect is still a challenge in the development of cosmetic agents. Both the repair effect and the protective effect are not ideal in the case of cosmetic compositions of the prior art due to many factors. For cleaning compositions of keratin fibers, a few raw materials are known which show a sustainable effect on strengthening and protecting the hair structure.

### SUMMARY

The object of the invention is to provide a cosmetic agent which prevents or reduces a damaging effect on keratin fibers. Furthermore, the object of the invention is to provide a cosmetic agent which exerts a repair effect on keratin fibers. The cosmetic agent should also develop an excellent cleaning effect.

The object of the invention is achieved by the subject matter of claim 1. A first subject matter of the invention is therefore a cosmetic agent for treating a keratin material, in particular for treating human hair, comprising
0.001 to 20 wt. % lactobionic acid in relation to the total weight of the cosmetic agent, and
0.01 to 40 wt. % of at least one anionic, amphoteric and/or non-ionic surfactant(s) in relation to the total weight of the cosmetic agent.

### DETAILED DESCRIPTION

In the context of the present invention, “keratin material” is to be understood in particular as human hair.

Lactobionic acid is 4-O-β-galactopyranosyl-D-gluconic acid which represents a saccharic acid. It is a disaccharide formed from gluconic acid and galactose. It can be formed by oxidation of lactose. In the context of the present invention, lactobionic acid is to be understood as meaning the free acid and also the salt. If the lactobionic acid is used as a salt, any cosmetically acceptable cation is used as a counterion. The weight fraction is then calculated in relation to the free acid.

The uniquely structured sugar acid, lactobionic acid, which is applied to the hair formulated in a hair cleaning composition, offers surprisingly good advantages, both with respect to the performance level and with respect to the multi-faceted effects. The lactobionic acid has structural stabilizing properties, strengthens the internal hair structure, offers color protection, limits structural damage to the hair fiber and generally has conditioning properties.

Furthermore, lactobionic acid has the following advantageous properties: With regard to its chemical properties, lactobionic acid has good solubility properties in water and is therefore versatile in its application in cosmetic agents. It can be advantageously used in surfactant-based emulsions, as in the present invention, but can also be incorporated in “leave-on” applications. Due to the structure, lactobionic acid has a high affinity for the hair fiber and therefore has advantageous effects in terms of hair care, repair effect and color protection.

According to a preferred embodiment, the cosmetic agent comprises in relation to the total weight of the cosmetic agent, 0.01 to 10 wt. %, more preferably 0.05 to 7, even more preferably 0.1 to 5 wt. %, lactobionic acid.

According to a further preferred embodiment, the cosmetic agent comprises in relation to the total weight of the cosmetic agent, 1 to 30 wt. %, more preferably 3 to 20 wt. %, even more preferably 6 to 15 wt. %, anionic, amphoteric and/or non-ionic surfactants. Within the scope of the present application, the following applies as a rule: If the cosmetic agent contains more than one surfactant, the percentages by weight of the surfactants are added. Thus, if the cosmetic agent contains anionic, amphoteric and/or nonionic surfactants, the anionic, amphoteric and/or nonionic surfactants are added.

If the anionic, amphoteric and/or non-ionic surfactant is restricted in the cosmetic agent of the present invention or if multiple surfactants are restricted, the anionic, amphoteric and/or non-ionic surfactant consists of the limited selection of the surfactants.

According to preferred embodiments, the cosmetic agent contains an anionic and a non-ionic surfactant or an anionic and an amphoteric surfactant or an amphoteric surfactant and a non-ionic surfactant. Of course, in the context of the present application, the formulation “a surfactant” always also includes multiple surfactants. Thus, for example, two or more anionic surfactants, two or more amphoteric surfactants or two or more non-ionic surfactants can also be contained in the cosmetic agent. Two or more surfactants of a kind can also be combined with two or more surfactants of another type. Thus, for example, two or more amphoteric surfactants can be combined with one or more anionic surfactants. If multiple surfactants, that is to say also multiple non-ionic, or multiple amphoteric, or multiple non-ionic and amphoteric surfactants, or multiple cationic and non-ionic surfactants, or multiple cationic and amphoteric surfactants are contained in the cosmetic agent, the quantities in percentage by weight always refer to the sum of the surfactants.

The cosmetic agent according to the preferred embodiment comprises the non-ionic surfactant selected from the group consisting of C₆- to C₁₈—, preferably C₈- to C₁₆, more preferably C₁₀- to C₁₄-alkyl polyglucosides, preferably cocoglucoside or decyl glucoside, polyglycerylalkylates, preferably PEG-7 glyceryl cocoate; cocamide MEA; and alkyl glycol ether from C₈- to C₂₄—, preferably C₁₂- to C₁₈-alkyl groups and 1 to 8, preferably 2 to 5, ethylene oxide units.

If the non-ionic surfactant is an alkyl polyglucoside, this is preferably selected from C₆- to C₁₈—, more preferably C₈- to C₁₆—, most preferably C₁₀- to C₁₄-alkyl polyglucosides. It is particularly preferred if the alkyl polyglucoside is cocoglucoside or decyl glucoside.

A further group of preferred nonionic surfactants is that of the polyglycerylalkylates. These are polyglycerol addition products to fatty acid esters, fatty acid alkanolamides or fatty acid glucamides, or they are ethylene oxide and polyglycerol addition products to fatty acid esters, fatty acid alkanolamides or fatty acid glucamides. The PEG-7 glyceryl cocoate in particular is preferred.

Furthermore, the non-ionic surfactants according to a preferred embodiment are alkyl glycol ethers which consist of a C₈- to C₂₄—, preferably C₁₂- to C₁₈-alkyl group and 1 to 8, preferably 2 to 5, ethylene oxide units.

According to preferred embodiments, the cosmetic agents contain 0.3 to 10 wt. %, more preferably 0.5 to 8 wt. %, even more preferably 0.75 to 6 wt. %, and in particular 1 to 5 wt. % non-ionic surfactant(s).

The cosmetic agents may contain an amphoteric surfactant. Surfactants that have both a negatively-charged functional group and a positively-charged functional group are referred to as amphoteric surfactants or zwitterionic surfactants.

According to preferred embodiments, the cosmetic agent contains an amphoteric surfactant, preferably a C₈₋₁₈-, more preferably C₁₀₋₁₆-alkylamido-(C₁₋₄-alkyl) betaine, a C₈₋₁₈-, more preferably C₁₀₋₁₆-alkylamido-(C₁₋₄-alkyl) sultaine, a C₁₀₋₁₈-alkyl amphoacetate or a C₁₀₋₁₈-alkyl amphodiacetate. In particular, according to preferred embodiments, the amphoteric surfactant is selected from the group consisting of cocamidopropyl betaines, amphoacetate, preferably disodium amphodiacetate, cocobetaines and mixtures thereof. Cocamidopropyl betaine is a particularly preferred fatty acid amide derivative which is particularly suitable as an amphoteric surfactant.

Suitable zwitterionic surfactants are further the so-called betaines, such as the N-alkyl-N,N-dimethylammonium glycinates, for example cocoalkyl dimethylammonium glycinate; the N-acylaminopropyl-N,N-dimethylammonium glycinates, for example cocoacylaminopropyl dimethylammonium glycinate; and 2-alkyl-3-carboxymethyl-S-hydroxyethyl imidazolines, each having 8 to 18 C atoms in the alkyl or acyl group, and cocoacylaminoethyl hydroxyethyl carboxymethyl glycinate. Further examples of suitable ampholytic surfactants are N-alkylglycines, N-alkylaminopropionic acids, N-alkylaminobutyric acids, N-alkyliminodipropionic acids, N-hydroxyethyl-N-alkyl amidopropyl glycines, N-alkyl taurines, N-alkyl sarcosines, 2-alkylaminopropionic acids, and alkylaminoacetic acids having in each case about 8 to 24 C atoms in the alkyl group. Particularly preferred amphoteric surfactants are N-coco-alkylaminopropionate, coco-acylaminoethylaminopropionate, and C₁₂-C₁₈-acyl sarcosine.

According to preferred embodiments, the cosmetic agents contain 0.3 to 20 wt. %, preferably 0.5 to 8 wt. %, more preferably 0.75 to 6 wt. %, and in particular 1 to 5 wt. % amphoteric surfactant(s).

Suitable anionic surfactants for the cosmetic agents are substances comprising an anionic group selected from the group consisting of carboxylate, sulfate, sulfonate and phosphate and comprising a linear or branched alkyl group comprising 8 to 30, more preferably 12 to 22, even more preferably 14 to 18, C atoms. Preferred anionic surfactants are acyl glutamates, acyl isethionates, acyl sarcosinates, and acyl taurates, each having a linear or branched acyl group with 6 to 22 carbon atoms and 0, 1, 2 or 3 double bonds, which group is selected in particularly preferred embodiments from an octanoyl, decanoyl, lauroyl, myristoyl, palmitoyl, and stearoyl group; esters of tartaric acid, citric acid, or succinic acid or the salts of these acids with alkylated glucose, in particular the products with the INCI designation disodium coco-glucoside citrate, sodium coco-glucoside tartrate and disodium coco-glucoside sulfosuccinate; alkyl polyglycol ether sulfates and ether carboxylic acids having 8 to 18 C atoms in the alkyl group and up to 12 ethoxy groups in the molecule; sulfosuccinic acid mono and dialkyl esters having 8 to 18 C atoms in the alkyl group; and sulfosuccinic acid monoalkyl polyoxyethyl esters having 8 to 18 C atoms in the alkyl group and 1 to 6 ethoxy groups.

Further preferred anionic surfactants are alkyl sulfates, alkyl polyglycol ether sulfates and ether carboxylic acid salts having 10 to 18 C atoms in the alkyl group and up to 12 glycol ether groups in the molecule, and sulfosuccinic acid mono- and dialkyl esters having 8 to 18 C atoms in the alkyl group, and sulfosuccinic acid monoalkyl polyoxyethyl esters having 8 to 18 C atoms in the alkyl group and 1 to 6 oxyethyl groups. Particularly preferred anionic surfactants are the alkali or ammonium salts of lauryl ether sulfate having a degree of ethoxylation of from 2 to 4 EO.

According to a preferred embodiment, the cosmetic agent comprises two, three or more different surfactants, the cosmetic agent preferably comprising one or two non-ionic surfactants selected from a C₆- to C₁₈-alkyl polyglucoside and/or a polyglycerylalkylate, and one or two amphoteric surfactants which are selected from amophoacetates and/or betaines. According to a further preferred embodiment, the cosmetic agent comprises one or two anionic surfactants selected from alkyl (ether) sulfates and/or alkyl (ether) sulfonates, and one or two amphoteric surfactants selected from amophoacetates and/or betaines.

According to preferred embodiments, the cosmetic agent contains one or more further care components. According to preferred embodiments, these are selected from the group consisting of a silicone, preferably an aminofunctional silicone or an alkoxylated dimethicone, more preferably an ethoxylated or propoxylated dimethicone, a polyol, a protein hydrolysate, preferably the hydrolysates of keratin, a proteolipid or a protein, preferably a vegetable protein, such as soy protein, pea protein or wheat protein.

According to a preferred embodiment, the cosmetic agent comprises a further care component selected from the group consisting of
a protein hydrolysate, preferably the hydrolysates of keratin, silk protein or wheat protein,
a protein,
a proteolipid, and
a cationic polymer.

The preferred proteolipid in the cosmetic agent is a proteolipid of formula I

R′—X—R″ (I),
where
R′ represents a straight-chain or branched, saturated or unsaturated hydrocarbon group having 11 to 24 carbon atoms,
R″ means a protein, a peptide or a protein hydrolysate,
X represents —C(O)O— or —N⁺(R^{III}2)-R^{IV}— or —N(R^{III})R^{IV}— or —C(O)—N(R^{V})R^{VI}—,
R^{III} means —(CH₂)ₓ—CH₃, where x=0-22, and
R^{IV} means —CH₂—CH(OH)—CH₂— or —(CH₂)ₓ—, where x=0-22;
R^{V} and R^{VI} represent, independently of one another, —H or —(CH₂)ₓ—CH₃, where x=0-22; with the proviso that R″ represents keratin or a keratin hydrolysate when X represents —C(O)O.

According to a preferred embodiment, the cosmetic agent comprises a cationic polymer. The cationic polymer is preferably selected from the group consisting of
a cationic cellulose polymer, preferably polyquaternium-10 and/or polyquaternium-24 and/or polyquaternium-67 and/or polyquaternium-72;
cationic guar derivative,
chitosan,
cationic honey,
polyquaternium-37,
polymeric dimethyldiallylammonium salts and copolymers thereof with esters and amides of acrylic acid and methacrylic acid and mixtures thereof.

The cationic polymer is preferably contained in the cosmetic agent in an amount of 0 to 10 wt. %, more preferably 0.01 to 8 wt. %, even more preferably 0.2 to 5 wt. % in relation to the total weight of the cosmetic agent.

### Preferred Silicones are Described Below:

Particularly preferred silicones are silicones of formula Si-I

(CH₃)₃Si—[O—Si(CH₃)₂]ₓ—O—Si(CH₃)₃ (Si—I),

in which x represents a number from 0 to 100, preferably from 0 to 50, more preferably from 0 to 20, and in particular 0 to 10. Preferred silicones that can be used within the context of the present invention have viscosities at 20° C. of 0.2 to 2 mm²s⁻¹, silicones having viscosities of 0.5 to 1 mm²s⁻¹ being particularly preferred.

Particularly preferred aminofunctional silicones are those of formula Si-II

M(RₐQ_{b}SiO_{(4-a-b)/2)x}(R_{c}SiO_{(4-c)/2)y}M (Si-II),

wherein, in the above formula, R is a hydrocarbon or a hydrocarbon group having 1 to approximately 6 carbon atoms, Q is a polar group of general formula —R¹HZ, in which R¹ is a bivalent, linking group bonded to hydrogen and the group Z, composed of carbon and hydrogen atoms, carbon, hydrogen and oxygen atoms, or carbon, hydrogen and nitrogen atoms, and Z is an organic, aminofunctional group containing at least one aminofunctional group; “a” assumes values in a range of from approximately 0 to approximately 2, “b” assumes values in a range of from approximately 1 to approximately 3, “a”+“b” is less than or equal to 3, and “c” is a number in a range of from approximately 1 to approximately 3, and x is a number in a range of from 1 to approximately 2,000, preferably from approximately 3 to approximately 50, and most preferably from approximately 3 to approximately 25, and y is a number in a range of from approximately 20 to approximately 10,000, preferably from approximately 125 to approximately 10,000, and most preferably from approximately 150 to approximately 1,000, and M is a suitable silicone end group as is known in the prior art, preferably trimethylsiloxy. Non-limiting examples of the groups represented by R include alkyl groups, such as methyl, ethyl, propyl, isopropyl, isopropyl, butyl, isobutyl, amyl, isoamyl, hexyl, isohexyl, and the like; alkenyl groups such as vinyl, halovinyl, alkylvinyl, allyl, haloallyl, and alkylallyl; cycloalkyl groups such as cyclobutyl, cyclopentyl, cyclohexyl, and the like; phenyl groups, benzyl groups, halohydrocarbon groups such as 3-chloropropyl, 4-bromobutyl, 3,3,3-trifluoropropyl, chlorocyclohexyl, bromophenyl, chlorophenyl, and the like, and sulfur-containing groups such as mercaptoethyl, mercaptopropyl, mercaptohexyl, mercaptophenyl, and the like; R is preferably an alkyl group, containing 1 to about 6 carbon atoms, and most preferably R is methyl. Examples of R¹ include methylene, ethylene, propylene, hexamethylene, decamethylene, —CH₂CH(CH₃) CH₂—, phenylene, naphthylene, —CH₂CH₂SCH₂CH₂—, —CH₂CH₂OCH₂—, —OCH₂CH₂CH₂—, —CH₂CH(CH₃)C(O)OCH₂—, —OCH₂CH₂—, (CH₂)₃CC(O)OCH₂CH₂—, —C₆H₄C₆H₄—, —C₆H₄CH₂C₆H₄—; and —(CH₂)₃C(O)SCH₂CH₂—.

Z is an organic, amino-functional group containing at least one functional amino group. A possible formula for Z is NH(CH₂)_{Z}NH₂, where z is 1 or more. Another possible formula for Z is-NH(CH₂)_{Z}(CH₂)_{ZZ}NH, where both z and zz are independently 1 or more, this structure comprising diamino ring structures such as piperazinyl. Z is most preferably a —NHCH₂CH₂NH₂ group. Another possible formula for Z is N(CH₂)_{Z}(CH₂)_{ZZ}NX₂ or —NX₂, wherein each X of X₂ is selected independently from the group consisting of hydrogen and alkyl groups having 1 to 12 carbon atoms, and zz is 0.

Q is most preferably a polar, amino-functional group of the formula —CH₂CH₂CH₂NHCH₂CH₂NH₂. In the formulas, “a” assumes values in the range of about 0 to about 2, “b” assumes values in the range of about 2 to about 3, “a”+ “b” is less than or equal to 3, and “c” is a number in the range of about 1 to about 3. The molar ratio of the RₐQ_{b} SiO_{(4-a-b)/2}-units to the R_{c}SiO_{(4-c)/2}-units are in the range of about 1:2 to 1:65, preferably from about 1:5 to about 1:65 and most preferably from about 1:15 to about 1:20. If one or more silicones of the above formula are used, then the various variable substituents in the above formula can be different in the various silicone components, present in the silicone mixture.

According to a preferred embodiment, the cosmetic agent contains polyols as a further care component. Polyols are polyhydric alcohols. These are predominantly well tolerated by the skin and as solvents ensure that the cosmetic agents obtainable therewith are not too solid or too heavy or too slow to dissolve. These advantageous properties arise particularly in the case of glycerol.

As polyhydric alcohols alditols, such as mannitol, isomalt, lactitol, sorbitol and xylitol, threitol, erythritol and arabitol, 1,2-propylene glycol, 1,3-butylene glycol, dipropylene glycol, glycerol or diglycerol, more preferably 1,2-propylene glycol, 1,3-butylene glycol, dipropylene glycol or glycerol, are preferably contained in the cosmetic agent. The polyhydric alcohols, in particular 1,2-propylene glycol, 1,3-butylene glycol, dipropylene glycol and glycerol, are preferably contained in the cosmetic agent in amounts of from 2 to 30 wt. %, more preferably from 5 to 15 wt. %.

As further preferred cosmetic active ingredients, oils are used. The cosmetic agent preferably comprises a mineral oil or a vegetable oil. The mineral oil can be selected from alkanes, in particular from an alkane mixture which has two or more alkanes that differ from one another by the number of C atoms. The preferably used plant oil is either modified or unmodified. Preferred vegetable oils are apricot seed oil, argan oil, jojoba oil, marula oil, almond oil, olive oil, coconut oil and/or sunflower oil.

The cosmetic agent contains shea butter (INCI name: butyrospermum parkii (shea) butter) or argan oil as very particularly preferred cosmetic active ingredients. The cosmetic agent contains shea butter (INCI name: butyrospermum parkii (shea) butter) and argan oil as very particularly preferred cosmetic active ingredients.

According to preferred embodiments, the cosmetic agent contains the further care components in relation to the total weight of the cosmetic agent in amounts of 0.01 to 15 wt. %, more preferably in amounts of 0.05 to 10 wt. %, even more preferably in amounts of 0.15 to 5 wt. %, in the cosmetic agent. These preferred amounts relate to the further care components insofar as this is not stated otherwise—for example in the polyol. Selected further care components may be contained in the cosmetic agent in other preferred amounts.

According to a further preferred embodiment, a cosmetic agent is provided in which the pH of the cosmetic agent is at pH 1 to pH 7, preferably pH 2 to pH 6, more preferably at pH 2.5 to 5.

Particularly preferred embodiments of the present invention are described below:

According to a preferred embodiment, the cosmetic agent for treating a keratin material, in particular for treating human hair, comprises a) 0.001 to 20 wt. % lactobionic acid in relation to the total weight of the cosmetic agent, and b) 1 to 30 wt. %, preferably 3 to 20 wt. %, more preferably 6 to 15 wt. %, an anionic and non-ionic surfactant in relation to the total weight of the cosmetic agent, wherein the percentages by weight of anionic and non-ionic surfactants are added.

According to a preferred embodiment, the cosmetic agent for treating a keratin material, in particular for treating human hair, a) comprises 0.001 to 20 wt. % lactobionic acid in relation to the total weight of the cosmetic agent, and b) 1 to 30 wt. %, preferably 3 to 20 wt. %, more preferably 6 to 15 wt. %, of an anionic and amphoteric surfactant in relation to the total weight of the cosmetic agent, wherein the percentages by weight of anionic and amphoteric surfactants are added.

According to a preferred embodiment, the cosmetic agent for treating a keratin material, in particular for treating human hair, comprises a) 0.001 to 20 wt. % lactobionic acid in relation to the total weight of the cosmetic agent, and b) 1 to 30 wt. %, preferably 3 to 20 wt. %, more preferably 6 to 15 wt. %, an anionic, amphoteric and non-ionic surfactant in relation to the total weight of the cosmetic agent, wherein the percentages by weight of anionic, amphoteric and non-ionic surfactants are added.

According to a preferred embodiment, the cosmetic agent for treating a keratin material, in particular for treating human hair, comprises a) 0.001 to 20 wt. % lactobionic acid in relation to the total weight of the cosmetic agent, and b) 1 to 30 wt. %, preferably 3 to 20 wt. %, more preferably 6 to 15 wt. %, an anionic, amphoteric and non-ionic surfactant in relation to the total weight of the cosmetic agent, and c) 0.0001 to 3 wt. % protein hydrolysate or proteolipid, in relation to the total weight of the cosmetic agent, wherein the percentages by weight of anionic, amphoteric and non-ionic surfactants are added.

The object of the invention is further achieved by the subject matter of claim 9. A second subject matter of the present invention is therefore a method for treating a keratin material, in particular for treating human hair, in which a cosmetic agent according to the invention is applied to the keratin material, massaged in and then rinsed with water.

The method represents a cleaning method of keratin fibers, in particular human hair.

The object of the invention is further achieved by the subject matter of claim 10. A third subject matter of the present invention is therefore the use of the cosmetic agent according to the invention for performing a repair effect, as an agent for strengthening the keratin fibers after damage or as an agent for color protection. The cosmetic agent according to the invention is preferably used for performing a repair effect, as an agent for strengthening the keratin fibers after damage or as an agent for color protection, for reducing or preventing harmful effects due to chemical treatments of the keratin material, due to air and water impurities, due to free radicals or due to UV radiation.

The cosmetic agent according to the invention is preferably used as a shampoo. This also means that the cosmetic agent preferably is a shampoo.

Features relating to preferred embodiments of the first subject matter of the invention, which are described above only in this regard, naturally apply accordingly to the second and third objects as features of preferred embodiments.

The following examples are intended to explain the subject of the present invention, without limiting it in any manner.

### Examples

### Shampoo:

| Ingredient | 1.1 | 1.2 | 1.3 | 1.4 | V |
|---|---|---|---|---|---|
| Sodium laureth | 10.0 | 10.0 | 12.0 | 12.0 | 10.0 |
| sulfate | | | | | |
| Ammonium | 4.0 | 4.0 | 2.0 | 2.0 | 4.0 |
| lauryl sulfate | | | | | |
| Cocoamidopropyl | 4.0 | | 4.0 | | 4.0 |
| betaine | | | | | |
| Cocobetaine | | 4.0 | | 4.0 | |
| Lactobionic acid | 1.0 | 1.0 | 1.0 | 1.0 | — |
| Cocamide MEA | 0.8 | 0.8 | 0.8 | 0.8 | 0.8 |
| PEG-7 glyceryl | 1.0 | 1.0 | 1.0 | 1.0 | 1.0 |
| cocoate | | | | | |
| Nicotinamide | 0.3 | 0.3 | 0.3 | 0.3 | 0.3 |
| Citric acid | 0.8 | 0.8 | 0.8 | 0.8 | 0.8 |
| Hydrolyzed | 0.3 | 0.3 | 0.3 | 0.3 | 0.3 |
| keratin | | | | | |
| (ProSina) | | | | | |
| Panthenol | 0.5 | 0.5 | 0.5 | 0.5 | 0.5 |
| Sodium chloride | 1.3 | 1.3 | 1.3 | 1.3 | 1.3 |
| Preservatives, | q.s. | q.s. | q.s. | q.s. | q.s. |
| perfume | | | | | |
| Water | up | up | up | up | up |
| | to 100 | to 100 | to 100 | to 100 | to 100 |

| Ingredient | 2.1 | 2.2 | 2.3 | 2.4 |
|---|---|---|---|---|
| Sodium laureth sulfate | 10.0 | 10.0 | 12.0 | 12.0 |
| Ammonium lauryl sulfate | 4.0 | 4.0 | 2.0 | 2.0 |
| Cocoamidopropyl betaine | 4.0 | | 4.0 | |
| Disodium | | 4.0 | | 4.0 |
| amphodiacetates | | | | |
| Lactobionic acid | 1.0 | 1.0 | 1.0 | 1.0 |
| Cocamide MEA | 0.8 | 0.8 | 0.8 | 0.8 |
| Decyl glucoside | 1.0 | 1.0 | 1.0 | 1.0 |
| Nicotinamide | 0.3 | 0.3 | 0.3 | 0.3 |
| Citric acid | 0.8 | 0.8 | 0.8 | 0.8 |
| Hydrolyzed keratin | 0.3 | 0.3 | 0.3 | 0.3 |
| (ProSina) | | | | |
| Panthenol | 0.5 | 0.5 | 0.5 | 0.5 |
| Sodium chloride | 1.3 | 1.3 | 1.3 | 1.3 |
| Preservatives, perfume | q.s. | q.s. | q.s. | q.s. |
| Water | up to 100 | up to 100 | up to 100 | up to 100 |

| Ingredient | Wt. % |
|---|---|
| C14-16 alpha olefin sulfonate, sodium salt | 8.0 |
| Cocoamidopropyl betaine | 4.0 |
| Sodium methyl cocoyl taurate | 4.0 |
| Guar hydroxypropyltrimonium chloride | 0.3 |
| Lanthanum chlorides | 0.15 |
| PEG-7 glyceryl cocoate | 1.0 |
| Hydrolyzed keratin | 0.3 |
| Citric acid | 0.5 |
| Lactobionic acid | 0.5 |
| PEG-120 methyl glucose dioleate | 0.7 |
| Sodium chloride | 1.3 |
| Water, preservative and optionally perfume | up to 100 |
| oils | |

1. A cosmetic agent for treating a keratin material, wherein the cosmetic agent comprises:
a) 0.001 to 20 wt. % of lactobionic acid in relation to the total weight of the cosmetic agent; and
b) 0.01 to 40 wt. % of at least one surfactant comprising an anionic surfactant, amphoteric surfactant, non-ionic surfactant, or combinations thereof, in relation to the total weight of the cosmetic agent, wherein the percentages by weight of the at least one surfactant comprising the anionic surfactant, amphoteric surfactant, non-ionic surfactant, or combinations thereof are added.

2. The cosmetic agent according to claim 1, wherein the cosmetic agent comprises, in relation to the total weight of the cosmetic agent, 0.01 to 10 wt. % of lactobionic acid, and
wherein the cosmetic agent comprises, in relation to the total weight of the cosmetic agent, 1 to 30 wt. % of the at least one surfactant.

3. The cosmetic agent according to claim 1, wherein the at least one surfactant is a non-ionic surfactant selected from the group consisting of C₆- to C₁₈-, polyglycerylalkylates; cocamide MEA; alkyl glycol ethers made of C₈- to C₂₄ alkyl groups and 1 to 8 ethylene oxide units, or combinations thereof.

4. The cosmetic agent according to claim 1, wherein the at least one surfactant is an amphoteric surfactant that is a C₈₋₁₈alkylamido-(C₁₋₄-alkyl) betaine, a C₈₋₁₈ alkylamido-(C₁₋₄-alkyl) sultaine, a C₁₀₋₁₈-alkyl amphoacetate, a C₁₀₋₁₈-alkyl amphoacetate, or combinations thereof.

5. The cosmetic agent according to claim 1, wherein the at least one surfactant is an anionic surfactant selected from the group consisting of alkyl(ether)sulfates from a C₈₋₁₈ alkyl group having 1 to 6 ethylene oxide units; alkyl(ether)sulfonates from a C₈₋₁₈ alkyl group having 1 to 6 ethylene oxide units; C₁₀₋₁₈ acyl isethionates; C₁₀₋₁₈ alkyl succinates; C₁₀₋₁₈ acyl taurates; and mixtures thereof.

6. The cosmetic agent according to claim 1, wherein the at least one surfactant comprises two or more different surfactants.

7. The cosmetic agent according to claim 1, further comprising a further care component selected from the group comprising:
a protein hydrolysate, a protein, a proteolipid, a cationic polymer, or combinations thereof.

8. The cosmetic agent according to claim 7, wherein the further care component is the proteolipid of formula I
R′—X—R″ (I),
wherein:
R′ represents a straight-chain or branched, saturated or unsaturated hydrocarbon group having 11 to 24 carbon atoms,
R″ represents a protein, a peptide or a protein hydrolysate,
X represents-C(O)O— or —N⁺(R^{III}₂)—R^{IV}— or —N(R^{III})R^{IV}— or —C(O)—N(R^{V})R^{VI}—,
R^{III} means —(CH₂)ₓ—CH₃, where x=0-22, and
R^{IV} means —CH₂—CH(OH)—CH₂- or —(CH₂)ₓ—, where x=0-22;
R^{V} and R^{VI} represent, independently of one another, —H or —(CH₂)ₓ—CH₃, where x=0-22;
wherein R″ represents keratin or a keratin hydrolysate when X represents —C(O)O,
and/or wherein the further care component is the cationic polymer selected from the group consisting of:
a cationic cellulose polymer,
cationic guar derivative,
chitosan,
cationic honey,
polyquaternium-37,
polymeric dimethyldiallylammonium salts and copolymers thereof with esters and amides of acrylic acid and methacrylic acid, and mixtures thereof.

9. A method for treating a keratin material, wherein the method comprises:
applying the cosmetic agent according to claim 1 to the keratin material; and
rinsing out the cosmetic agent with water.

10. (canceled)
